(19) United States
(12) Patent Application Publication
(10) Pub. No.: US 2026/0029242 A1
(43) Pub. Date: Jan. 29, 2026

(54) DRIVING ASSISTANCE APPARATUS, DRIVING ASSISTANCE METHOD, STORAGE MEDIUM, AND DRIVING ASSISTANCE SYSTEM

(71) Applicant: HONDA MOTOR CO., LTD., Tokyo (JP)

(72) Inventors: Satoru Shinkawa, Wako-shi (JP); Shigenobu Mitsuzawa, Wako-shi (JP); Hiroshi Ono, Wako-shi (JP); Hiroki Taniguchi, Wako-shi (JP); Nozomi Nagatani, Wako-shi (JP)

(21) Appl. No.: 19/212,970

(22) Filed: May 20, 2025

(30) Foreign Application Priority Data
Jul. 26, 2024 (JP) 2024-121014

(51) Int. Cl. G01C 21/34; A61B 5/00; A61B 5/0205; A61B 5/024; A61B 5/16; A61B 5/18; B60Q 9/00

(52) U.S. Cl. CPC G01C 21/3484; A61B 5/0205; A61B 5/165; A61B 5/18; A61B 5/4815; B60Q 9/00; G01C 21/3461; A61B 5/024; A61B 2503/22

(57) ABSTRACT

A smartphone includes: a biological information acquisition unit that acquires biological information about a driver; a driving behavior acquisition unit that acquires driving behavior information indicating driving behavior of the driver; a danger level evaluation unit that evaluates a driving danger level indicating a danger degree while the driver is driving a vehicle, based on the biological information and the driving behavior information; an area setting unit that sets a danger area in map information when the driving danger level is equal to or higher than a predetermined value; a position acquisition unit that acquires a travel position of the vehicle on a map; and a notification unit that outputs, when the travel position approaches the danger area, a first notification indicating that the travel position has approached the danger area.

### INCORPORATION BY REFERENCE

The present application claims priority under 35 U.S.C. § 119 to Japanese Patent Application No. 2024-121014 filed on Jul. 26, 2024. The content of the application is incorporated herein by reference in its entirety.

### BACKGROUND OF THE INVENTION

### Field of the Invention

The present invention relates to a driving assistance apparatus, a driving assistance method, a storage medium, and a driving assistance system.

### Description of the Related Art

There has been proposed an apparatus that acquires biological information about a driver and driving information relating to a driving operation of the driver, and determines appropriateness of the driver's driving based on these acquired pieces of information (for example, see Japanese Patent Laid-Open No. 2023-145468).

In recent years, efforts have gained momentum for providing access to sustainable transport systems that also take into account people in vulnerable situations among traffic participants. Towards this end, efforts are focused on research and development for further improving traffic safety, convenience, and the like through research and development relating to a driver assistance system that supports the safety and health of drivers.

However, in the driver assistance system, a problem is to further improve traffic safety, convenience, and the like by appropriately notifying a user of a danger area set on a map in accordance with a driving danger level.

In order to solve the above problem, an object of the present application is to output a notification indicating that a travel position has approached a danger area set on a map, when the driving danger level is equal to or higher than a predetermined value. Thus, the present application contributes to development of a sustainable transportation system.

### SUMMARY OF THE INVENTION

According to a first aspect for achieving the above object, a driving assistance apparatus includes: a biological information acquisition unit configured to acquire biological information about a user; a driving behavior acquisition unit configured to acquire driving behavior information indicating driving behavior of the user; a danger level evaluation unit configured to evaluate a driving danger level indicating a danger degree while the user is driving a vehicle, based on the biological information and the driving behavior information; a map information storage unit configured to store map information; an area setting unit configured to set a danger area in the map information when the driving danger level is equal to or higher than a predetermined value; a position acquisition unit configured to acquire a travel position of the vehicle on a map, based on position information of the vehicle; and a notification unit configured to output, when the travel position approaches the danger area, a first notification indicating that the travel position has approached the danger area.

According to a second aspect for achieving the above object, a driving assistance method includes: a biological information acquisition step of acquiring biological information about a user; a driving behavior acquisition step of acquiring driving behavior information indicating driving behavior of the user; a danger level evaluation step of evaluating a driving danger level indicating a danger degree while the user is driving a vehicle, based on the biological information and the driving behavior information; an area setting step of setting a danger area in map information stored in advance, when the driving danger level is equal to or higher than a predetermined value; a position acquisition step of acquiring a travel position of the vehicle on a map, based on position information of the vehicle; and a notification step of outputting, when the travel position approaches the danger area, a first notification indicating that the travel position has approached the danger area.

According to a third aspect for achieving the above object, a non-transitory computer readable storage medium storing a driving assistance program causes a processor included in a driving assistance apparatus to function as: a biological information acquisition unit configured to acquire biological information about a user; a driving behavior acquisition unit configured to acquire driving behavior information indicating driving behavior of the user; a danger level evaluation unit configured to evaluate a driving danger level indicating a danger degree while the user is driving a vehicle, based on the biological information and the driving behavior information; a map information storage unit configured to store map information; an area setting unit configured to set a danger area in the map information when the driving danger level is equal to or higher than a predetermined value; a position acquisition unit configured to acquire a travel position of the vehicle on a map, based on position information of the vehicle; and a notification unit configured to output, when the travel position approaches the danger area, a first notification indicating that the travel position has approached the danger area.

According to a fourth aspect for achieving the above object, a driving assistance system includes: a biological detection apparatus configured to detect biological information about a user; a driving detection apparatus configured to detect driving behavior of the user; and a driving assistance apparatus configured to communicate with the biological detection apparatus and the driving detection apparatus, in which the driving assistance apparatus includes: a biological information acquisition unit configured to acquire the biological information about the user from the biological detection apparatus; a driving behavior acquisition unit configured to acquire driving behavior information indicating the driving behavior of the user from the driving detection apparatus; a danger level evaluation unit configured to evaluate a driving danger level indicating a danger degree while the user is driving a vehicle, based on the biological information and the driving behavior information; a map information storage unit configured to store map information; an area setting unit configured to set a danger area in the map information when the driving danger level is equal to or higher than a predetermined value; a position acquisition unit configured to acquire a travel position of the vehicle on a map, based on position information of the vehicle; and a notification unit configured to output, when the travel position approaches the danger area, a first notification indicating that the travel position has approached the danger area.

According to the driving assistance apparatus, the driving assistance method, the storage medium, and the driving assistance system, it is possible to notify the user that the travel position has approached the danger area set on the map, when the driving danger level is equal to or higher than the predetermined value.

### BRIEF DESCRIPTION OF THE DRAWINGS

FIG. 1 is a diagram showing an example of a configuration of a driving assistance system;

FIG. 2 is a diagram showing an example of a configuration of a smartwatch;

FIG. 3 is a diagram showing an example of a configuration of an in-vehicle apparatus;

FIG. 4 is a diagram showing an example of the configuration of the in-vehicle apparatus;

FIG. 5 is a diagram showing an example of a configuration of a smartphone;

FIG. 6 is a diagram showing an example of correlation between biological information, a health rating, and advice information;

FIG. 7 is a screen diagram showing an example of a display screen displayed on the smartphone; and

FIG. 8 is a flowchart showing an example of processing of the smartphone.

### DETAILED DESCRIPTION OF THE INVENTION

Hereinafter, embodiments of a driving assistance apparatus, a driving assistance method, a driving assistance program, and a driving assistance system will be described with reference to the drawings.

### 1. Configuration of Notification Control System

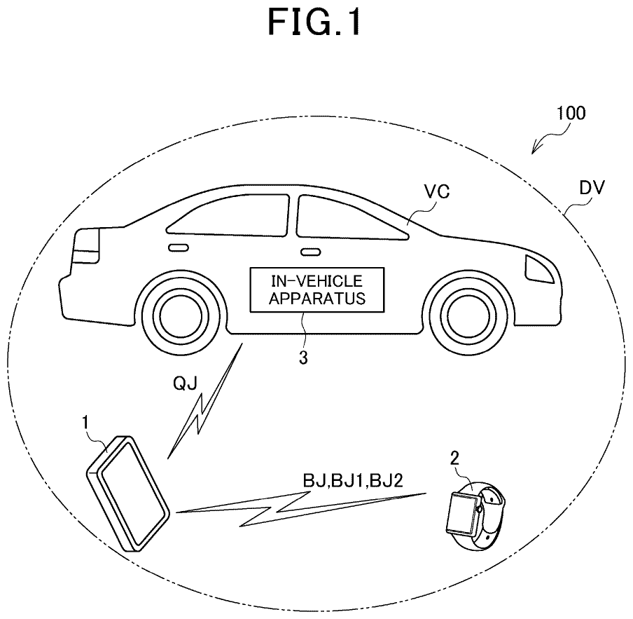

First, a configuration of a driving assistance system 100 will be described with reference to FIG. 1.

FIG. 1 is a diagram showing an example of the configuration of the driving assistance system 100.

As shown in FIG. 1, the driving assistance system 100 includes a smartphone 1, a smartwatch 2, and an in-vehicle apparatus 3. Note that a dash-dotted line in FIG. 1 indicates that the smartphone 1, the smartwatch 2, and a vehicle VC are property of a driver DV. The driver DV drives the vehicle VC.

The driver DV corresponds to an example of a “user”.

The smartphone 1 wirelessly communicates with the smartwatch 2 and the in-vehicle apparatus 3 in accordance with, for example, a communication standard such as Wi-Fi®.

Note that the smartphone 1 may wirelessly communicate with the smartwatch 2 and the in-vehicle apparatus 3 through, for example, a communication method supporting a communication standard such as Bluetooth®, Bluetooth Low Energy (BLE), Wi-Fi®, or Zigbee®.

The smartphone 1 acquires biological information BJ about the driver DV from the smartwatch 2. The smartphone 1 acquires driving behavior information QJ from the in-vehicle apparatus 3.

The smartphone 1 calculates a driving danger level DR indicating a danger degree while the driver DV is driving the vehicle VC, based on the biological information BJ and the driving behavior information QJ. The smartphone 1 sets a danger area DA in map information MP when the driving danger level DR is equal to or higher than a predetermined value. When a travel position PS of the vehicle VC approaches the danger area DA, the smartphone 1 outputs a first notification NT1 indicating that the travel position PS has approached the danger area DA.

The smartphone 1 corresponds to an example of the “driving assistance apparatus”.

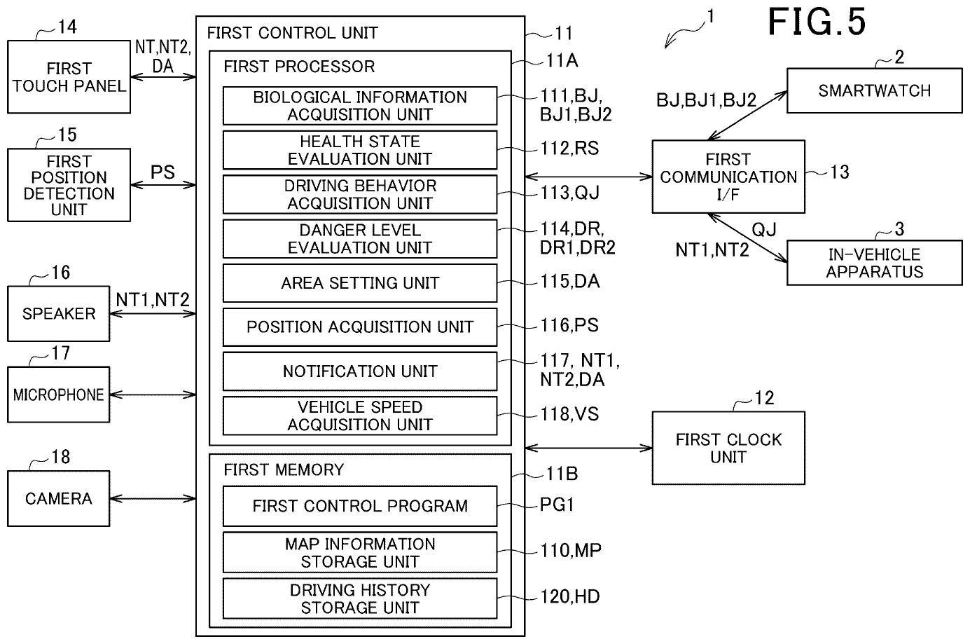

The smartphone 1 will be described further with reference to FIGS. 5 to 8.

The smartwatch 2 is worn on a wrist of the driver DV. The smartwatch 2 generates the biological information BJ. The biological information BJ includes, for example, a heart rate of the driver DV.

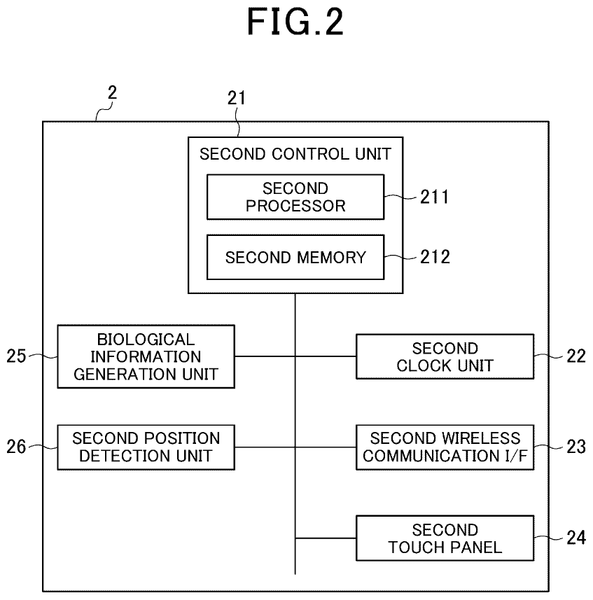

The smartwatch 2 will be described further with reference to FIG. 2.

The smartwatch 2 corresponds to an example of a “biological detection apparatus”.

The in-vehicle apparatus 3 is installed in the vehicle VC. The in-vehicle apparatus 3 generates the driving behavior information QJ. The driving behavior information QJ includes operation information from when the driver DV has driven the vehicle VC.

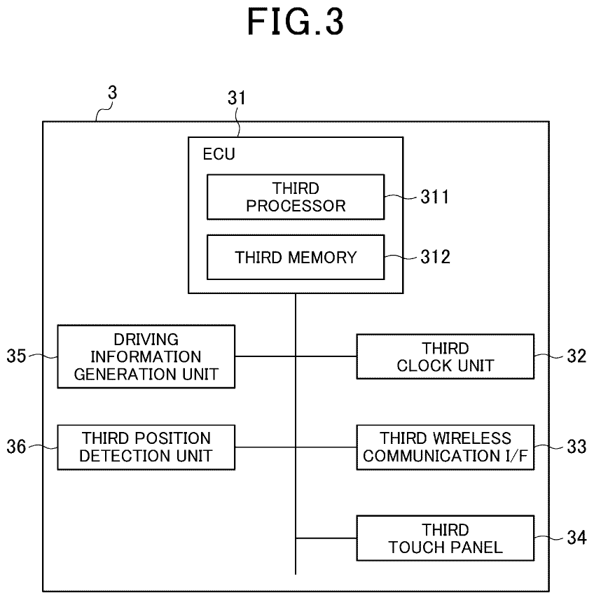

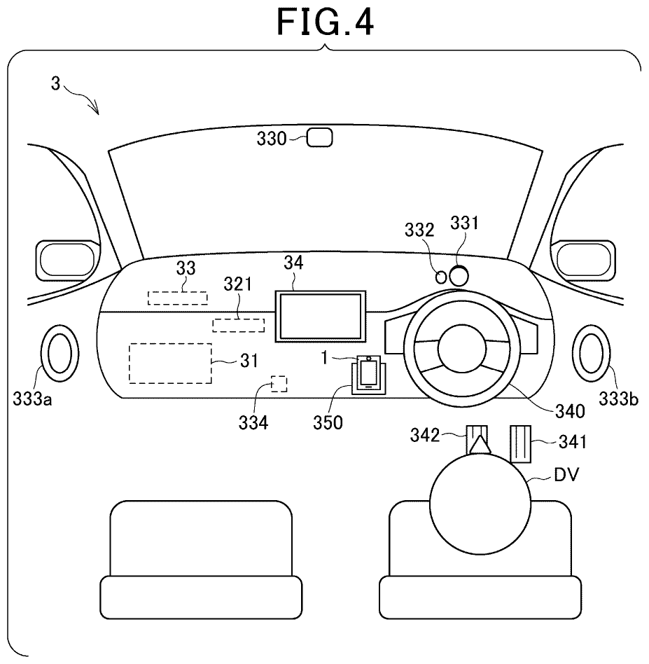

The in-vehicle apparatus 3 will be described further with reference to FIGS. 3 and 4.

The in-vehicle apparatus 3 corresponds to an example of a “driving detection apparatus”.

### 2. Configuration of Smartwatch

Next, a configuration of the smartwatch 2 will be described with reference to FIG. 2. FIG. 2 is a diagram showing an example of the configuration of the smartwatch 2.

As shown in FIG. 2, the smartwatch 2 includes a second control unit 21, a second clock unit 22, a second wireless communication interface 23, a second touch panel 24, a biological information generation unit 25, and a second position detection unit 26.

The second control unit 21 controls each unit of the smartwatch 2. The second control unit 21 includes a second processor 211 and a second memory 212.

The second memory 212 consists of, for example, a non-volatile semiconductor memory, or volatile and non-volatile semiconductor memories. The second processor 211 consists of a central processing unit (CPU), and microprocessor unit (MPU), or the like. The second processor 211 may consist of a single processor or can also consist of a plurality of processors.

The second processor 211 controls each unit of the smartwatch 2 by executing a second control program stored in the second memory 212.

The second clock unit 22 has a clock function of measuring a date and time. The second clock unit 22 acquires date and time information from a server apparatus (not illustrated). The second clock unit 22 adjusts the clock function by using the acquired date and time information. The second clock unit 22 displays the date and time information on the second touch panel 24.

The second wireless communication interface 23 includes an antenna that receives a wireless signal and an interface circuit that processes the wireless signal received by the antenna. The second wireless communication interface 23 performs wireless communication with external apparatuses including the server apparatus (not illustrated) and the smartphone 1.

The second touch panel 24 includes a display panel and a touch sensor. The display panel consists of a liquid crystal panel, an organic electroluminescent (EL) panel, or the like. The touch sensor detects a touch operation of the driver DV on the display panel.

The second touch panel 24 displays an image on the display panel in accordance with an instruction from the second control unit 21. The second touch panel 24 detects a touch operation of the driver DV on the display panel.

The biological information generation unit 25 is a computer (ECU) including a processor and a memory, and generates the biological information BJ.

The biological information generation unit 25 measures, at preset intervals, the biological information BJ that is the biological information about the driver DV who is wearing the smartwatch 2.

The biological information BJ includes first biological information BJ1 and second biological information BJ2. The first biological information BJ1 is biological information about the driver DV while the driver DV is driving. The second biological information BJ2 is biological information about the driver DV in a first period before the driver DV starts driving. The first period is, for example, three months. The first period may be, for example, half a year.

The first period is set depending on how stable a health state of the driver DV is. For example, the first period is preferably set to be longer the more stable the health state of the driver DV is.

The first biological information BJ1 includes, for example, first sleep information BJ11, first exercise information BJ12, and first stress information BJ13. The second biological information BJ2 includes, for example, second sleep information BJ21, second exercise information BJ22, and second stress information BJ23.

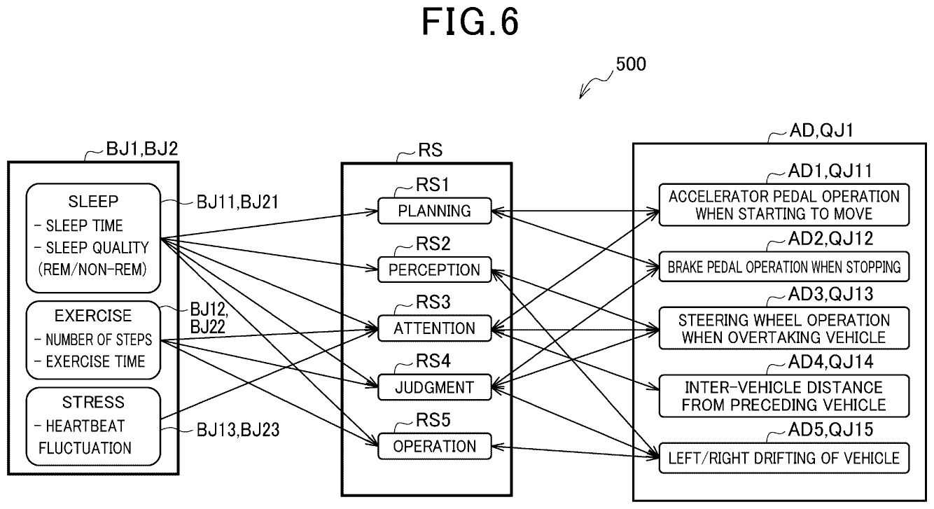

The first sleep information BJ11, the first exercise information BJ12, the first stress information BJ13, the second sleep information BJ21, the second exercise information BJ22, and the second stress information BJ23 will be described further with reference to FIGS. 5 and 6.

The biological information generation unit 25 outputs the measured biological information BJ to the second control unit 21. The second control unit 21 transmits the biological information BJ to the smartphone 1 via the second wireless communication interface 23.

The second position detection unit 26 includes a global navigation satellite system (GNSS) sensor. The second position detection unit 26 measures a position, that is, a latitude, a longitude, an altitude, and the like of the smartwatch 2, by receiving a positioning signal transmitted from a GNSS satellite. The second position detection unit 26 outputs position information indicating the measured position to the second control unit 21.

### 3. Configuration of In-Vehicle Apparatus

Next, a configuration of the in-vehicle apparatus 3 will be described with reference to FIGS. 3 and 4. FIG. 3 is a diagram showing an example of the configuration of the in-vehicle apparatus 3.

As shown in FIG. 3, the in-vehicle apparatus 3 includes an electronic control unit (ECU) 31, a third clock unit 32, a third wireless communication interface 33, a third touch panel 34, a driving information generation unit 35, and a third position detection unit 36.

The ECU 31 controls each unit of the in-vehicle apparatus 3. The ECU 31 includes a third processor 311 and a third memory 312.

The third memory 312 consists of, for example, a non-volatile semiconductor memory, volatile and non-volatile semiconductor memories, or the like. The third processor 311 consists of a CPU, an MPU, or the like. The third processor 311 may consist of a single processor or can also consist of a plurality of processors.

The third processor 311 controls each unit of the in-vehicle apparatus 3 by executing a third control program stored in the third memory 312.

The third clock unit 32 has a clock function of measuring the date and time. The third clock unit 32 acquires the date and time information from the server apparatus (not illustrated). The third clock unit 32 adjusts the clock function by using the acquired date and time information. The third clock unit 32 displays the date and time information on the third touch panel 34.

The third wireless communication interface 33 includes an antenna that receives a wireless signal and an interface circuit that processes the wireless signal received by the antenna. The third wireless communication interface 33 performs wireless communication with external apparatuses including the server apparatus (not illustrated) and the smartphone 1.

The third touch panel 34 includes a display panel and a touch sensor. The display panel consists of a liquid crystal panel, an organic EL panel, or the like. The touch sensor detects a touch operation of the driver DV on the display panel.

The third touch panel 34 displays an image on the display panel in accordance with an instruction from the ECU 31. The third touch panel 34 detects a touch operation of the driver DV on the display panel.

The driving information generation unit 35 is a computer (ECU) including a processor and a memory, and generates the driving behavior information QJ.

The driving behavior information QJ includes first driving behavior information QJ1 and second driving behavior information QJ2. The first driving behavior information QJ1 indicates driving behavior of the driver DV while driving the vehicle VC. The second driving behavior information QJ2 indicates driving behavior in a second period before the driver DV starts driving the vehicle VC. The second period is, for example, three months. The second period may be, for example, half a year.

The second period is set depending on how often the driver DV drives the vehicle VC. The second period is preferably set to be shorter the more the driver DV drives the vehicle VC.

The driving information generation unit 35 acquires the driving behavior information QJ from various sensors disposed in the vehicle VC. The driving behavior information QJ includes, for example, vehicle speed information, acceleration information, accelerator operation information, brake operation information, and steering wheel operation information.

Note that the driving behavior information QJ may include road information about a road on which the vehicle VC travels. The road information includes, for example, construction information or traffic congestion information.

The vehicle speed information indicates a travel speed of the vehicle VC. The acceleration information indicates an acceleration detected by an acceleration sensor. The acceleration sensor detects, for example, an acceleration in an up-down direction, an acceleration in a front-rear direction, and an acceleration in a left-right direction of the vehicle VC.

The accelerator operation information indicates an operation state of an acceleration pedal 341 caused by the driver DV. The accelerator operation information includes, for example, a pressed amount and a pressing speed of the acceleration pedal 341.

The brake operation information indicates an operation state of a brake pedal 342 caused by the driver DV. The brake operation information includes, for example, a pressed amount and a pressing speed of the brake pedal 342.

The steering wheel operation information indicates an operation state of a steering wheel 340 caused by the driver DV. The steering wheel operation information includes, for example, a steering angle and a change speed of the steering angle of the steering wheel 340.

The acceleration pedal 341, the brake pedal 342, and the steering wheel 340 will be described further with reference to FIG. 4.

The driving information generation unit 35 outputs the driving behavior information QJ acquired from the various sensors to the ECU 31. The ECU 31 transmits the driving behavior information QJ to the smartphone 1 via the third wireless communication interface 33.

The third position detection unit 36 includes a GNSS sensor. The third position detection unit 36 measures a position, that is, a latitude, a longitude, an altitude, and the like of the vehicle VC, by receiving a positioning signal transmitted from a GNSS satellite. The third position detection unit 36 outputs position information indicating the measured position to the ECU 31.

Next, the configuration of the in-vehicle apparatus 3 will be further described with reference to FIG. 4. FIG. 4 is a diagram showing an example of the configuration of the in-vehicle apparatus 3. FIG. 4 is a view when looking forward from a top portion direction of a driver seat in the vehicle VC.

As shown in FIG. 4, the steering wheel 340 described with reference to FIG. 3 is disposed, for example, in front of the driver DV. The acceleration pedal 341 and the brake pedal 342 described with reference to FIG. 3 are disposed, for example, at the feet of the driver DV. The third touch panel 34 described with reference to FIG. 3 is, for example, to the left of the steering wheel 340 and is disposed substantially at the center of a dashboard. The ECU 31 described with reference to FIG. 3 is disposed, for example, in the dashboard in front of a passenger seat. The third wireless communication interface 33 described with reference to FIG. 3 is, for example, disposed in the dashboard in front of the passenger seat.

Note that illustration of the third clock unit 32, the driving information generation unit 35, and the third position detection unit 36 described with reference to FIG. 3 is omitted in FIG. 4.

As shown in FIG. 4, the in-vehicle apparatus 3 further includes a navigation apparatus 321, a front camera 330, a driver camera 331, a microphone 332, a left speaker 333a, a right speaker 333b, an acceleration sensor 334, and a holder 350.

The navigation apparatus 321 includes the third position detection unit 36 described in reference with FIG. 3 and displays, for example, the position on a map of the vehicle VC on the third touch panel 34. The navigation apparatus 321 is disposed in the dashboard in front of the passenger seat.

The front camera 330 captures a front view of the vehicle VC. The front camera 330 includes an image sensor such as a charge-coupled device (CCD) or a complementary metal-oxide-semiconductor (CMOS). The front camera 330 is disposed on an inner side of a top portion of a front window.

The driver camera 331 images the driver DV and generates a captured image. The driver camera 331 includes an image sensor such as a CCD or a CMOS. The image of the driver includes, for example, at least a head portion of the driver.

The driver camera 331 outputs the generated image of the driver to the ECU 31.

The microphone 332 detects sound produced by the driver DV and generates a detection signal. The sound includes speech produced by the driver DV.

The microphone 332 outputs the generated detection signal to the ECU 31.

The left speaker 333a is disposed on an inner side of a left door of the vehicle VC. The right speaker 333b is disposed on an inner side of a right door of the vehicle VC. The left speaker 333a and the right speaker 333b output speech. For example, the left speaker 333a and the right speaker 333b may output speech indicating advice information AD.

The acceleration sensor 334 detects, for example, the acceleration in the up-down direction, the front-rear direction, and the left-right direction of the vehicle VC. The acceleration sensor 334 is disposed, for example, inside the dashboard.

The holder 350 fixably supports the smartphone 1 to the dashboard. When the driver DV is driving the vehicle VC, the driver DV places the smartphone 1 in the holder 350. The holder 350 is disposed, for example, on the dashboard in front of the driver seat to the left.

### 4. Configuration of Smartphone

Next, a configuration of the smartphone 1 will be described with reference to FIG. 5. FIG. 5 is a diagram showing an example of the configuration of the smartphone 1.

The smartphone 1 includes a first control unit 11, a first clock unit 12, a first wireless communication interface 13, a first touch panel 14, a first position detection unit 15, a speaker 16, a microphone 17, and a camera 18.

The first control unit 11 controls each unit of the smartphone 1. The first control unit 11 includes a first processor 11A and a first memory 11B.

The first memory 11B consists of, for example, a non-volatile semiconductor memory, or volatile and non-volatile semiconductor memories. The first processor 11A consists of a CPU, an MPU, or the like. The first processor 11A may consist of a single processor or can also consist of a plurality of processors.

The first processor 11A controls each unit of the smartphone 1 by executing a first control program PG1 stored in the first memory 11B.

The first clock unit 12 has a clock function of measuring the date and time. The first clock unit 12 acquires the date and time information from the server apparatus (not illustrated). The first clock unit 12 adjusts the clock function by using the acquired date and time information. The first clock unit 12 displays the date and time information on the first touch panel 14.

The first wireless communication interface 13 includes an antenna that receives a wireless signal and an interface circuit that processes the wireless signal received by the antenna. The first wireless communication interface 13 performs wireless communication with external apparatuses including the server apparatus (not illustrated), the smartwatch 2, and the in-vehicle apparatus 3.

The first touch panel 14 includes a display panel and a touch sensor. The display panel consists of a liquid crystal panel, an organic EL panel, or the like. The touch sensor detects a touch operation of the driver DV on the display panel.

The first touch panel 14 displays an image on the display panel in accordance with an instruction from the first control unit 11. The first touch panel 14 displays, for example, an image indicating the advice information AD on the display panel in accordance with an instruction from the first control unit 11.

The first touch panel 14 detects a touch operation of the driver DV on the display panel.

The first position detection unit 15 includes a GNSS sensor. The first position detection unit 15 measures a position, that is, a latitude, a longitude, an altitude, and the like of the smartphone 1, by receiving a positioning signal transmitted from a GNSS satellite. The first position detection unit 15 outputs position information indicating the measured position to the first control unit 11.

The speaker 16 outputs various speech in accordance with an instruction from the first control unit 11. For example, the speaker 16 outputs speech indicating the advice information AD in accordance with an instruction from the first control unit 11.

The microphone 17 accepts an input of various speech in accordance with an instruction from the first control unit 11. The microphone 17 outputs the accepted speech to the first control unit 11.

The microphone 17 may accept, for example, speech from the driver DV.

The camera 18 captures various images in accordance with an instruction from the first control unit 11. The camera 18 includes an image sensor such as a CCD or a CMOS. The camera 18 outputs the generated images to the first control unit 11.

The first control unit 11 includes a biological information acquisition unit 111, a health state evaluation unit 112, a driving behavior acquisition unit 113, a danger level evaluation unit 114, an area setting unit 115, a position acquisition unit 116, a notification unit 117, a vehicle speed acquisition unit 118, a map information storage unit 110, and a driving history storage unit 120.

To be specific, the first processor 11A functions as the biological information acquisition unit 111, the health state evaluation unit 112, the driving behavior acquisition unit 113, the danger level evaluation unit 114, the area setting unit 115, the position acquisition unit 116, the notification unit 117, and the vehicle speed acquisition unit 118 by executing the first control program PG1 stored in the first memory 11B. The first processor 11A causes the first memory 11B to function as the map information storage unit 110 and the driving history storage unit 120 by executing the first control program PG1 stored in the first memory 11B.

The first control program PG1 corresponds to an example of the “driving assistance program”.

The map information storage unit 110 stores the map information MP in advance.

For example, the first control unit 11 acquires the map information MP from the server apparatus (not illustrated) and causes the map information storage unit 110 to store the acquired map information. Note that the map information MP includes information relating to the danger area DA. The information relating to the danger area DA includes, for example, information indicating a position of the danger area DA in the map information, danger degree information DA1 indicating a danger degree in the danger area DA, and danger content information DA2 indicating a danger content in the danger area DA.

The driving history storage unit 120 stores a travel history HD of the vehicle VC of the driver DV.

The travel history HD includes, for example, route information indicating a route that the vehicle VC has traveled. The travel history HD also includes, for example, information indicating whether the driver DV has demonstrated appropriate driving behavior in the danger area DA included in the route. Appropriate driving behavior is, in other words, safe driving behavior.

The first memory 11B stores the biological information BJ and the driving behavior information QJ. The biological information BJ includes the first biological information BJ1 and the second biological information BJ2. The driving behavior information QJ includes the first driving behavior information QJ1 and the second driving behavior information QJ2.

Each of the first biological information BJ1 and the second biological information BJ2 is, for example, acquired from the smartwatch 2 by the biological information acquisition unit 111, and stored in the first memory 11B by the biological information acquisition unit 111.

Each of the first driving behavior information QJ1 and the second driving behavior information QJ2 is, for example, acquired from the in-vehicle apparatus 3 by the driving behavior acquisition unit 113, and stored in the first memory 11B by the driving behavior acquisition unit 113.

The biological information acquisition unit 111 acquires the biological information BJ indicating the biological information of the driver DV. The biological information BJ includes the first biological information BJ1 and the second biological information BJ2.

The first biological information BJ1 is the biological information about the driver DV while driving the vehicle VC. While driving the vehicle VC indicates a period from when the driver DV starts driving the vehicle VC to when the driver DV stops driving the vehicle VC. The biological information acquisition unit 111 acquires, for example, the first biological information BJ1 from the smartwatch 2. The first biological information BJ1 includes, for example, the first sleep information BJ11, the first exercise information BJ12, and the first stress information BJ13.

The first sleep information BJ11 indicates, for example, a drowsiness degree estimated from the second sleep information BJ21 to be described below. The first exercise information BJ12 indicates, for example, an exercise ability level estimated from the second exercise information BJ22 to be described below. The first stress information BJ13 indicates a stress degree experienced by the driver DV. The first stress information BJ13 includes heartbeat information and heartbeat fluctuation information. The heartbeat information indicates, for example, a heart rate per minute. The heartbeat fluctuation information indicates a state of a change in the heart rate.

The second biological information BJ2 is biological information about the driver DV in the first period before the driver DV starts driving the vehicle VC. The first period is, for example, three months. The first period may be, for example, half a year. The biological information acquisition unit 111 acquires, for example, the second biological information BJ2 from the smartwatch 2.

The second biological information BJ2 includes the second sleep information BJ21, the second exercise information BJ22, and the second stress information BJ23.

The second sleep information BJ21 includes, for example, sleep time information and information indicating sleep quality. The sleep time information indicates, for example, a sleep time per day of the driver DV. The information indicating sleep quality includes, for example, REM sleep time information and non-REM sleep time information per day of the driver DV.

The second exercise information BJ22 includes, for example, step number information and exercise time information. The step number information indicates, for example, a cumulative value of the number of steps per day of the driver DV. The exercise time information indicates, for example, a cumulative value of an exercise time per day of the driver DV.

The second stress information BJ23 includes heartbeat information and heartbeat fluctuation information. The heartbeat information indicates, for example, a heart rate per minute. The heartbeat fluctuation information indicates a state of a change in the heart rate.

The health state evaluation unit 112 evaluates the health state of the driver DV and generates a health rating RS indicating a healthiness degree of the driver DV, based on the first biological information BJ1 and the second biological information BJ2. The health rating RS corresponds to each of five abilities indicating a driving ability AB of the driver DV. The driving ability AB consists of, for example, a planning ability AB1, a perception ability AB2, an attention ability AB3, a judgment ability AB4, and an operation ability AB5.

The health rating RS consists of a planning ability rating RS1, a perception ability rating RS2, an attention ability rating RS3, a judgment ability rating RS4, and an operation ability rating RS5.

The planning ability rating RS1 indicates the health rating RS corresponding to the planning ability AB1. The planning ability AB1 is, for example, an ability of the driver DV to make a driving plan. The driving plan is, for example, a plan to cause the vehicle VC to travel from a parking position to a destination.

The perception ability rating RS2 indicates the health rating RS corresponding to the perception ability AB2. The perception ability AB2 is, for example, an ability to perceive a surrounding situation while the driver DV is driving.

The attention ability rating RS3 indicates the health rating RS corresponding to the attention ability AB3. The attention ability AB3 is, for example, an ability to pay attention to the surrounding situation in order to anticipate danger when the driver DV is driving.

The judgment ability rating RS4 indicates the health rating RS corresponding to the judgment ability AB4. The judgment ability AB4 is, for example, an ability to judge whether to perform any type of operation in accordance with the surrounding situation while the driver DV is driving.

The operation ability rating RS5 indicates the health rating RS corresponding to the operation ability AB5. The operation ability AB5 is, for example, an ability to perform an appropriate driving operation in accordance with the surrounding situation while the driver DV is driving.

The health state evaluation unit 112 attaches, for example, a score out of ten to each of the planning ability rating RS1, the perception ability rating RS2, the attention ability rating RS3, the judgment ability rating RS4, and the operation ability rating RS5.

The health state evaluation unit 112 attaches, for example, a score out of ten to the health rating RS as, for example, an average value of the planning ability rating RS1, the perception ability rating RS2, the attention ability rating RS3, the judgment ability rating RS4, and the operation ability rating RS5. The health state evaluation unit 112 attaches, for example, a score out of ten to the health rating RS as, for example, a weighted average value of the planning ability rating RS1, the perception ability rating RS2, the attention ability rating RS3, the judgment ability rating RS4, and the operation ability rating RS5.

The driving behavior acquisition unit 113 acquires the driving behavior information QJ indicating the driving behavior of the driver DV. The driving behavior information QJ includes the first driving behavior information QJ1 and the second driving behavior information QJ2.

The first driving behavior information QJ1 indicates the driving behavior of the driver DV while driving the vehicle VC. The driving behavior acquisition unit 113 acquires, for example, the first driving behavior information QJ1 from the in-vehicle apparatus 3. The first driving behavior information QJ1 includes, for example, the vehicle speed information, the acceleration information, the accelerator operation information, the brake operation information, and the steering wheel operation information.

The second driving behavior information QJ2 indicates the driving behavior in the second period before the driver DV starts driving the vehicle VC. The second period is, for example, three months. The second period may be, for example, half a year.

The driving behavior acquisition unit 113 acquires, for example, the second driving behavior information QJ2 from the in-vehicle apparatus 3. The second driving behavior information QJ2 includes, for example, the vehicle speed information, the acceleration information, the accelerator operation information, the brake operation information, and the steering wheel operation information.

The danger level evaluation unit 114 evaluates the driving danger level DR indicating the danger degree while the driver DV is driving the vehicle VC, based on the biological information BJ and the driving behavior information QJ.

The danger level evaluation unit 114 evaluates, for example, the driving danger level DR indicating the danger degree while the driver DV is driving the vehicle VC, based on the first biological information BJ1, the second biological information BJ2, the first driving behavior information QJ1, and the second driving behavior information QJ2.

The driving danger level DR includes, for example, a first driving danger level DR1 and a second driving danger level DR2.

The first driving danger level DR1 indicates the danger degree while driving, based on the health state of the driver DV. The second driving danger level DR2 indicates the danger degree while driving, based on the driving behavior information QJ.

The danger level evaluation unit 114 calculates, for example, the driving danger level DR from the first driving danger level DR1 and the second driving danger level DR2.

The danger level evaluation unit 114 evaluates, for example, the first driving danger level DR1 based on the health rating RS to which the health state evaluation unit 112 has attached a score. The higher the health rating RS is, the lower the danger level evaluation unit 114 evaluates the first driving danger level DR1 at. The danger level evaluation unit 114 calculates, for example, the first driving danger level DR1 using the following formula (1):

$$DR 1 = 10 - RS \qquad ( 1 )$$

The danger level evaluation unit 114 evaluates, for example, the second driving danger level DR2 based on the first driving behavior information QJ1 and the second driving behavior information QJ2.

The danger level evaluation unit 114 generates, for example, a reference driving property indicating a property of the driving behavior of the driver DV, based on the second driving behavior information QJ2. The danger level evaluation unit 114 evaluates a variability degree in the reference driving property of the driving behavior indicated by the first driving behavior information QJ1 acquired by the driving behavior acquisition unit 113 in the current driving. The danger level evaluation unit 114 evaluates the second driving danger level DR2 indicating the danger degree while the driver DV is driving the vehicle VC, based on the variability degree. The danger level evaluation unit 114 evaluates, for example, the second driving danger level DR2 out of ten.

For example, a case will be described in which the driving behavior is an accelerator operation when the driver DV starts moving the vehicle VC.

The danger level evaluation unit 114 generates, for example, a reference driving property indicating a property of the accelerator operation of the driver DV when the driver DV starts moving the vehicle VC, based on an accelerator operation when starting the vehicle VC of past driving by the driver DV included in the second driving behavior information QJ2. The reference driving property is, for example, an acceleration distribution indicating a frequency distribution of an average value of an acceleration caused by the accelerator operation when the driver DV starts moving the vehicle VC.

Next, the danger level evaluation unit 114 evaluates a variability degree in the acceleration distribution of the accelerator operation when the driver DV starts moving the vehicle VC indicated by the first driving behavior information QJ1 acquired by the driving behavior acquisition unit 113 in the current driving.

For example, the danger level evaluation unit 114 calculates the second driving danger level DR2 as “0” when the acceleration caused by the accelerator operation when the driver DV starts moving the vehicle VC in the current driving is included in a range of a first threshold value with respect to the average value of the acceleration distribution. The first threshold value is, for example, one times a standard deviation.

For example, the danger level evaluation unit 114 calculates the second driving danger level DR2 as “5” when the acceleration caused by the accelerator operation when the driver DV starts moving the vehicle VC in the current driving is outside the range of the first threshold value of the acceleration distribution and is included in a range of a second threshold value of the acceleration distribution with respect to the average value of the acceleration distribution. The range of the second threshold value is, for example, three times the standard deviation.

For example, the danger level evaluation unit 114 calculates the second driving danger level DR2 as “10” when the acceleration caused by the accelerator operation in the current driving is outside the range of the second threshold value of the acceleration distribution with respect to the average value of the acceleration distribution.

In this way, the danger level evaluation unit 114 calculates the second driving danger level DR2 of the accelerator operation indicated by the driving behavior information QJ acquired in the current driving.

The danger level evaluation unit 114 calculates the driving danger level DR from the first driving danger level DR1 and the second driving danger level DR2. The danger level evaluation unit 114 calculates, for example, the driving danger level DR out of ten as an average value of the first driving danger level DR1 and the second driving danger level DR2. For example, the danger level evaluation unit 114 calculates the driving danger level DR out of ten as a weighted average value of the first driving danger level DR1 and the second driving danger level DR2.

The area setting unit 115 sets the danger area DA in the map information when the driving danger level DR is equal to or higher than the predetermined value. The predetermined value is, for example, a score of “3”.

For example, the higher the driving danger level DR is, the more danger areas DA the area setting unit 115 sets in the map information.

Each of the plurality of danger areas DA is associated with the danger degree information DA1 indicating the danger degree in the danger area DA, and the danger content information DA2 indicating the danger content in the danger area DA.

The area setting unit 115 sets, for example, a predetermined number of danger areas DA in the map information when the driving danger level DR is the predetermined value. For example, in each of the predetermined number of danger areas DA, the danger degree indicated by the danger degree information DA1 is equal to or higher than a predetermined degree. The predetermined degree indicates, for example, that the danger degree is high.

The area setting unit 115 sets, for example, a first predetermined number, which is larger than the predetermined number, of danger areas DA in the map information when the driving danger level DR is equal to or higher than a first predetermined value, which is larger than the predetermined value. The first predetermined value is, for example, a score of “5”. For example, in each of the first predetermined number of danger areas DA, the danger degree indicated by the danger degree information DA1 is equal to or higher than a first predetermined degree. The first predetermined degree indicates, for example, that the danger degree is medium.

The area setting unit 115 sets, for example, a second predetermined number, which is larger than the first predetermined number, of danger areas DA in the map information when the driving danger level DR is equal to or higher than a second predetermined value, which is larger than the first predetermined value. The second predetermined value is, for example, a score of “7”. For example, in each of the second predetermined number of danger areas DA, the danger degree indicated by the danger degree information DA1 is equal to or higher than a second predetermined degree. The second predetermined degree indicates, for example, that the danger degree is low. In this case, the area setting unit 115 sets all predicted danger areas DA in the map information.

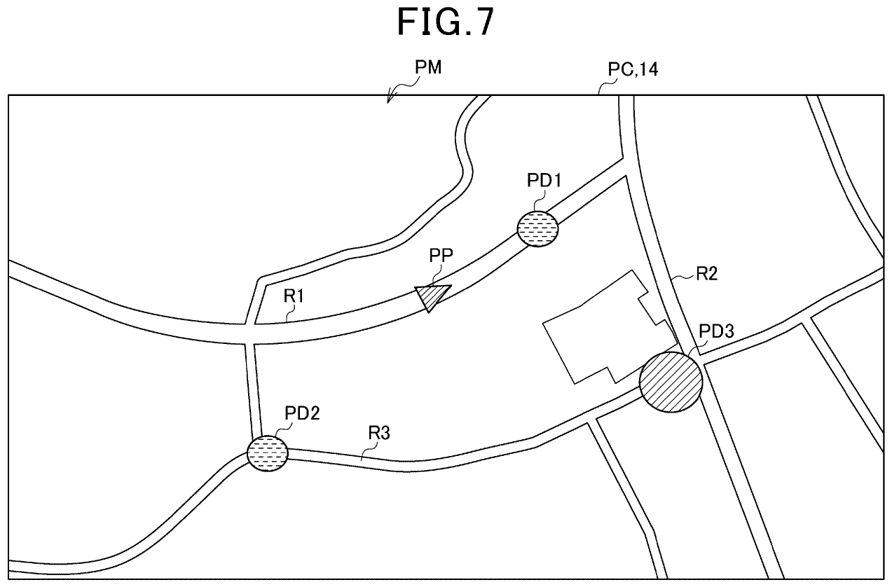

The danger area DA will be described further with reference to FIG. 7.

The position acquisition unit 116 acquires the travel position PS of the vehicle VC on the map, based on position information of the vehicle VC. The position acquisition unit 116 acquires, for example, the position information of the vehicle VC from the in-vehicle apparatus 3.

When the travel position PS approaches the danger area DA, the notification unit 117 outputs the first notification NT1 indicating that the travel position PS has approached the danger area DA. The first notification NT1 includes, for example, the danger content information DA2 corresponding to the danger area DA.

The first notification NT1 indicates, for example, “Rear-end collisions frequently occur ahead. Please keep sufficient distance between vehicles.”

The notification unit 117 outputs, for example, speech indicating the first notification NT1 from the speaker 16. The notification unit 117 may, for example, display an image indicating the first notification NT1 on the first touch panel 14.

The vehicle speed acquisition unit 118 acquires the vehicle speed information indicating a travel speed VS of the vehicle. The vehicle speed acquisition unit 118 acquires, for example, the vehicle speed information indicating the travel speed VS of the vehicle from the in-vehicle apparatus 3.

The notification unit 117 outputs a second notification NT2 being a more urgent (strong) notification than the first notification NT1, when the travel speed VS is higher than a reference speed VA of a road on which the vehicle VC is traveling.

The second notification NT2 indicates, for example, “Danger. Your vehicle speed is too high. Rear-end collisions frequently occur ahead. Please keep sufficient distance between vehicles.”

The notification unit 117 outputs, for example, speech indicating the second notification NT2 from the speaker 16. The notification unit 117 may, for example, display an image indicating the second notification NT2 on the first touch panel 14.

When the travel history HD indicates that the danger area DA has been traveled in the past and appropriate driving behavior has been demonstrated in the danger area DA, the notification unit 117 does not output the first notification NT1 relating to this danger area DA. Appropriate driving behavior is, for example, driving behavior that ensures safety.

The notification unit 117 displays the danger area DA on the map in a form that corresponds to the danger degree of the danger area DA. The danger degree corresponds, for example, to the danger degree information DA1.

For example, the higher the danger degree of the danger area DA is, the more the notification unit 117 highlights the danger area DA displayed on the map. For example, the higher the danger degree of the danger area DA is, the larger the notification unit 117 displays the danger area DA on the map.

The danger degree corresponds to an example of a “danger level”.

The notification unit 117 outputs, as the first notification NT1, the advice information AD relating to an ability category evaluated to be the lowest by the health state evaluation unit 112. Note that the driving ability AB consists of, for example, the planning ability AB1, the perception ability AB2, the attention ability AB3, the judgment ability AB4, and the operation ability AB5.

The advice information AD corresponding to each of the planning ability AB1, the perception ability AB2, the attention ability AB3, the judgment ability AB4, and the operation ability AB5 will be described with reference to FIG. 6.

When the travel position PS approaches the danger area DA, the notification unit 117 displays warning information relating to the danger area DA. When the travel position PS approaches the danger area DA, the notification unit 117 displays the warning information relating to the danger area DA on the first touch panel 14.

### 5. Correlation Between Biological Information, Health Rating, and Advice Information

Next, a correlative relationship between the biological information BJ, the health rating RS, and the advice information AD will be described with reference to FIG. 6. FIG. 6 is a diagram showing an example of a correlation between the biological information BJ, the health rating RS, and the advice information AD. The health rating RS corresponds to each of the five abilities indicating the driving ability AB. FIG. 6 illustrates a correlation diagram 500.

The correlation diagram 500 includes the biological information BJ, the health rating RS, and the advice information AD.

As shown in FIG. 6, the biological information BJ includes the first biological information BJ1 and the second biological information BJ2.

The first biological information BJ1 is biological information about the driver DV when the driver DV is driving. The first biological information BJ1 includes, for example, the first sleep information BJ11, the first exercise information BJ12, and the first stress information BJ13.

The second biological information BJ2 is biological information about the driver DV before the driver DV starts driving. The second biological information BJ2 includes, for example, the second sleep information BJ21, the second exercise information BJ22, and the second stress information BJ23.

The health rating RS includes the planning ability rating RS1, the perception ability rating RS2, the attention ability rating RS3, the judgment ability rating RS4, and the operation ability rating RS5.

The health state evaluation unit 112 evaluates the health state of the driver DV and generates the health rating RS indicating the healthiness degree of the driver DV, based on the second biological information BJ2. The health rating RS consists of the planning ability rating RS1, the perception ability rating RS2, the attention ability rating RS3, the judgment ability rating RS4, and the operation ability rating RS5.

Note that the health state evaluation unit 112 may correct the health rating RS by using the first biological information BJ1.

Arrows between the first biological information BJ1 and the health rating RS in FIG. 6 indicate correlations.

For example, the planning ability rating RS1 correlates with the second sleep information BJ21. Accordingly, the health state evaluation unit 112 generates the planning ability rating RS1 based on the second sleep information BJ21.

For example, the perception ability rating RS2 correlates with the second sleep information BJ21. Accordingly, the health state evaluation unit 112 generates the perception ability rating RS2 based on the second sleep information BJ21.

For example, the attention ability rating RS3 correlates with the second sleep information BJ21, the second exercise information BJ22, and the second stress information BJ23. Accordingly, the health state evaluation unit 112 generates the attention ability rating RS3 based on the second sleep information BJ21, the second exercise information BJ22, and the second stress information BJ23.

For example, the judgment ability rating RS4 correlates with the second sleep information BJ21 and the second exercise information BJ22. Accordingly, the health state evaluation unit 112 generates the judgment ability rating RS4 based on the second sleep information BJ21 and the second exercise information BJ22.

For example, the operation ability rating RS5 correlates with the second sleep information BJ21 and the second exercise information BJ22. Accordingly, the health state evaluation unit 112 generates the operation ability rating RS5 based on the second sleep information BJ21 and the second exercise information BJ22.

As shown in FIG. 6, the advice information AD includes, for example, first advice information AD1, second advice information AD2, third advice information AD3, fourth advice information AD4, and fifth advice information AD5.

The first driving behavior information QJ1 includes first behavior information QJ11, second behavior information QJ12, third behavior information QJ13, fourth behavior information QJ14, and fifth behavior information QJ15.

The first advice information AD1 is, for example, the advice information AD relating to an operation of the acceleration pedal 341 when the driver DV starts moving the vehicle VC. The first advice information AD1 is, for example, the advice information AD encouraging not to abruptly start moving.

The first behavior information QJ11 indicates an operation of the acceleration pedal 341 when the driver DV starts moving the vehicle VC. The notification unit 117 may, for example, cause the speaker 16 to output speech indicating the first advice information AD1, when the driving danger level DR is increased due to the first behavior information QJ11.

The second advice information AD2 is, for example, the advice information AD relating to an operation of the brake pedal 342 when the driver DV stops the vehicle VC. The second advice information AD2 is, for example, the advice information AD encouraging not to abruptly stop.

The second behavior information QJ12 indicates an operation of the brake pedal 342 when the driver DV stops the vehicle VC. The notification unit 117 may, for example, cause the speaker 16 to output speech indicating the second advice information AD2, when the driving danger level DR is increased due to the second behavior information QJ12.

The third advice information AD3 is, for example, the advice information AD relating to an operation of the steering wheel 340 when the driver DV causes the vehicle VC to overtake another vehicle. The third advice information AD3 is, for example, the advice information AD encouraging not to abruptly steer.

The third behavior information QJ13 indicates an operation of the steering wheel 340 when the driver DV causes the vehicle VC to overtake another vehicle. The notification unit 117 may, for example, cause the speaker 16 to output speech indicating the third advice information AD3, when the driving danger level DR is increased due to the third behavior information QJ13.

The fourth advice information AD4 is, for example, the advice information AD relating to an inter-vehicle distance between the vehicle VC that the driver DV is driving and another vehicle traveling in front. The fourth advice information AD4 is, for example, the advice information AD encouraging to keep an appropriate inter-vehicle distance.

The fourth behavior information QJ14 indicates an operation of changing the inter-vehicle distance between the vehicle VC that the driver DV is driving and another vehicle traveling in front. The fourth behavior information QJ14 includes, for example, an operation of the acceleration pedal 341 and an operation of the brake pedal 342. The notification unit 117 may, for example, cause the speaker 16 to output speech indicating the fourth advice information AD4, when the driving danger level DR is increased due to the fourth behavior information QJ14.

The fifth advice information AD5 is the advice information AD relating to drifting in the left-right direction of the vehicle VC that the driver DV is driving. The fifth advice information AD5 is, for example, the advice information AD encouraging to suppress drifting in the left-right direction of the vehicle VC.

The fifth behavior information QJ15 indicates an operation that causes drifting in the left-right direction of the vehicle VC that the driver DV is driving. The fifth behavior information QJ15 includes, for example, loosening of a grip state on the steering wheel 340. The notification unit 117 may, for example, cause the speaker 16 to output speech indicating the fifth advice information AD5, when the driving danger level DR is increased due to the fifth behavior information QJ15.

Arrows between the health rating RS and the advice information AD in FIG. 6 indicate correlations.

The notification unit 117 selects, for example, the advice information AD corresponding to a rating with the lowest score from among the planning ability rating RS1, the perception ability rating RS2, the attention ability rating RS3, the judgment ability rating RS4, and the operation ability rating RS5.

For example, as shown in FIG. 6, the planning ability rating RS1 correlates with the first advice information AD1 and the second advice information AD2. Accordingly, the notification unit 117 notifies the driver DV of, for example, the first advice information AD1 and the second advice information AD2 with priority, when the planning ability rating RS1 is the rating with the lowest score.

The perception ability rating RS2 correlates with the third advice information AD3 and the fifth advice information AD5. Accordingly, the notification unit 117 notifies the driver DV of, for example, the third advice information AD3 and the fifth advice information AD5 with priority, when the perception ability rating RS2 is the rating with the lowest score.

The attention ability rating RS3 correlates with the first advice information AD1, the third advice information AD3, and the fourth advice information AD4. Accordingly, the notification unit 117 notifies the driver DV of, for example, the first advice information AD1, the third advice information AD3, and the fourth advice information AD4 with priority, when the attention ability rating RS3 is the rating with the lowest score.

The judgment ability rating RS4 correlates with the second advice information AD2, the third advice information AD3, and the fifth advice information AD5. Accordingly, the notification unit 117 notifies the driver DV of, for example, the second advice information AD2, the third advice information AD3, and the fifth advice information AD5 with priority, when the judgment ability rating RS4 is the rating with the lowest score.

The operation ability rating RS5 correlates with the fifth advice information AD5. Accordingly, the notification unit 117 notifies the driver DV of, for example, the fifth advice information AD5 with priority, when the operation ability rating RS5 is the rating with the lowest score.

### 6. Display Screen

Next, a display screen PC displayed on the smartphone 1 will be described with reference to FIG. 7. FIG. 7 is a screen diagram showing an example of the display screen PC displayed on the smartphone 1. The display screen PC is displayed on the first touch panel 14 via the notification unit 117.

The display screen PC includes a map image PM corresponding to the map information MP; a first area image PD1, a second area image PD2, and a third area image PD3 corresponding to the danger area DA; and a position image PP indicating the travel position PS of the vehicle VC.

The map image PM includes a first road R1, a second road R2, and a third road R3. Each of the first road R1 and the third road R3 intersects the second road R2.

The first area image PD1 is disposed in front of the travel position PS of the vehicle VC indicated by position image PP. The second area image PD2 is disposed at a location on the third road R3 where a radius of curvature is small. The third area image PD3 is disposed on the third road R3 in front of the intersection with the second road R2.

The third area image PD3 is displayed larger than the first area image PD1 and the second area image PD2. Displaying the third area image PD3 in this way indicates that the danger degree of the danger area DA corresponding to the third area image PD3 is higher than the danger degree of the danger area DA corresponding to the first area image PD1 and the danger degree of the danger area DA corresponding to the second area image PD2.

### 7. Processing of Smartphone

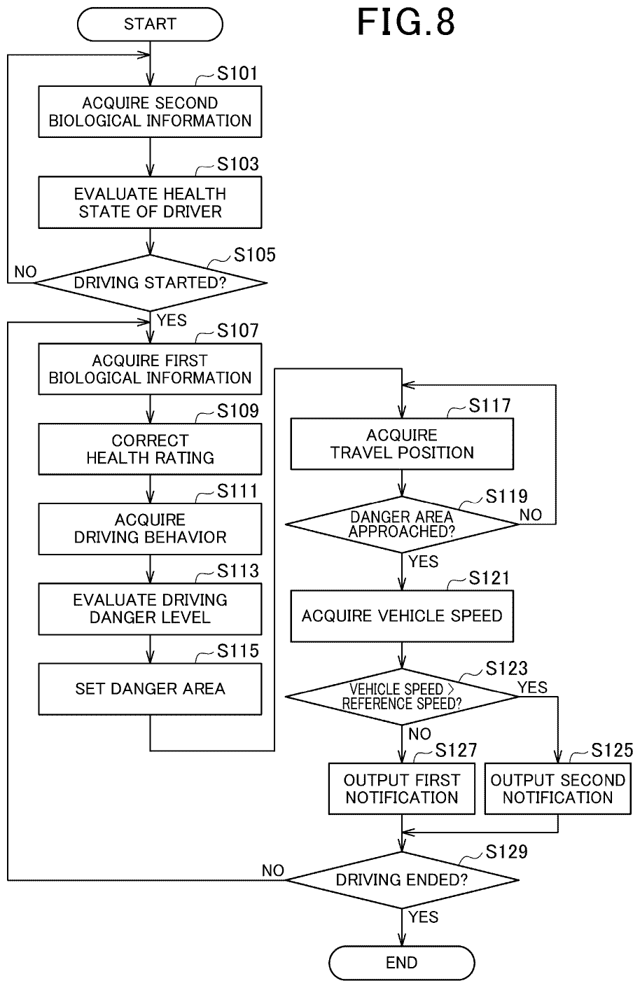

Next, processing of the smartphone 1 will be described with reference to FIG. 8. FIG. 8 is a flowchart showing an example of the processing of the smartphone 1.

First, in step S101, the biological information acquisition unit 111 acquires, for example, the second biological information BJ2 from the smartwatch 2. The second biological information BJ2 is biological information about the driver DV in the first period before the driver DV starts driving the vehicle VC.

Next, in step S103, the health state evaluation unit 112 evaluates the health state of the driver DV and generates the health rating RS indicating the healthiness degree of the driver DV, based on the second biological information BJ2.

Next, in step S105, the first control unit 11 determines whether the driver DV has started driving the vehicle VC. The first control unit 11 determines, for example, that the driver DV has started driving the vehicle VC when the smartphone 1 is placed in the holder 350.

When the first control unit 11 determines that the driver DV has not started driving the vehicle VC (NO in step S105), the processing returns to step S101. When the first control unit 11 determines that the driver DV has started driving the vehicle VC (YES in step S105), the processing proceeds to step S107.

In step S107, the biological information acquisition unit 111 acquires the first biological information BJ1 from the smartwatch 2.

Next, in step S109, the health state evaluation unit 112 corrects the health rating RS generated in step S103, based on the first biological information BJ1.

Next, in step S111, the driving behavior acquisition unit 113 acquires the driving behavior information QJ indicating the driving behavior of the driver DV from, for example, the in-vehicle apparatus 3. The driving behavior information QJ includes the first driving behavior information QJ1 and the second driving behavior information QJ2. The first driving behavior information QJ1 indicates the driving behavior of the driver DV while driving the vehicle VC. The second driving behavior information QJ2 indicates the driving behavior in the second period before the driver DV starts driving the vehicle VC.

Next, in step S113, the danger level evaluation unit 114 evaluates the driving danger level DR indicating the danger degree while the driver DV is driving the vehicle VC. The danger level evaluation unit 114 evaluates, for example, the driving danger level DR indicating the danger degree while the driver DV is driving the vehicle VC, based on the first biological information BJ1, the second biological information BJ2, the first driving behavior information QJ1, and the second driving behavior information QJ2.

Next, in step S115, the area setting unit 115 sets the danger area DA in the map information when the driving danger level DR is equal to or higher than the predetermined value. Hereinafter, a case will be described in which the driving danger level DR is equal to or higher than the predetermined value.

Next, in step S117, the position acquisition unit 116 acquires the travel position PS of the vehicle VC on the map from the in-vehicle apparatus 3, based on the position information of the vehicle VC.

Next, in step S119, the first control unit 11 determines whether the travel position PS has approached the danger area DA.

When the first control unit 11 determines that the travel position PS has not approached the danger area DA (NO in step S119), the processing returns to step S117. When the first control unit 11 determines that the travel position PS has approached the danger area DA (YES in step S119), the processing proceeds to step S121.

In step S121, the vehicle speed acquisition unit 118 acquires the vehicle speed information indicating the travel speed VS of the vehicle from the in-vehicle apparatus 3.

Next, in step S123, the first control unit 11 determines whether the travel speed VS is higher than the reference speed VA of the road on which the vehicle VC is traveling.

When the first control unit 11 determines that the travel speed VS is higher than the reference speed VA of the road on which the vehicle VC is traveling (YES in step S123), the processing proceeds to step S125.

In step S125, the notification unit 117 outputs the second notification NT2 being a more urgent notification than the first notification NT1. Subsequently, the processing proceeds to step S129.

When the first control unit 11 determines that the travel speed VS is not higher than the reference speed VA of the road on which the vehicle VC is traveling (NO in step S123), the processing proceeds to step S127.

In step S127, the notification unit 117 outputs the first notification NT1 indicating that the travel position PS has approached the danger area DA.

Next, in step S129, the first control unit 11 determines whether the driver DV has stopped driving the vehicle VC. The first control unit 11 determines, for example, that the driver DV has stopped driving the vehicle VC when the smartphone 1 is removed from the holder 350.

When the first control unit 11 determines that the driver DV has not stopped driving the vehicle VC (NO in step S129), the processing returns to step S107. When the first control unit 11 determines that the driver DV has stopped driving the vehicle VC (YES in step S129), the processing ends.

Step S107 corresponds to an example of a “biological information acquisition step”.

Step S111 corresponds to an example of a “driving behavior acquisition step”.

Step S113 corresponds to an example of a “danger level evaluation step”.

Step S115 corresponds to an example of an “area setting step”.

Step S117 corresponds to an example of a “position acquisition step”.

Step S127 corresponds to an example of a “notification step”.

### 8. Configurations and Effects

As described with reference to FIGS. 1 to 8, the smartphone 1 according to the present embodiment includes: the biological information acquisition unit 111 that acquires the biological information BJ about the driver DV; the driving behavior acquisition unit 113 that acquires the driving behavior information QJ indicating the driving behavior of the driver DV; the danger level evaluation unit 114 that evaluates the driving danger level DR indicating the danger degree while the driver DV is driving the vehicle VC, based on the biological information BJ and the driving behavior information QJ; the map information storage unit 110 that stores the map information MP; the area setting unit 115 that sets the danger area DA in the map information MP when the driving danger level DR is equal to or higher than the predetermined value; the position acquisition unit 116 that acquires the travel position PS of the vehicle VC on the map, based on the position information of the vehicle VC; and the notification unit 117 that outputs, when the travel position PS approaches the danger area DA, the first notification NT1 indicating that the travel position PS has approached the danger area DA.

Therefore, it is possible to appropriately set the danger area DA, since the danger area DA is set in the map information MP when the driving danger level DR is equal to or higher than the predetermined value. It is also possible to appropriately output the first notification NT1, since the first notification NT1 indicating that the travel position PS has approached the danger area DA is output when the travel position PS approaches the danger area DA.

In the smartphone 1, the vehicle speed acquisition unit 118 is provided that acquires the vehicle speed information indicating the travel speed VS of the vehicle VC, and the notification unit 117 outputs the second notification NT2 being a more urgent notification than the first notification NT1, when the travel speed VS is higher than the reference speed VA of the road on which the vehicle VC is traveling.

Therefore, it is possible to appropriately output the second notification NT2, since the second notification NT2 being a more urgent notification than the first notification NT1 is output when the travel speed VS is higher than the reference speed VA of the road on which the vehicle VC is traveling.

In the smartphone 1, the biological information BJ includes: the first biological information BJ1 being biological information about the driver DV while driving the vehicle VC and the second biological information BJ2 being biological information about the driver DV in the first period before the driver DV starts driving the vehicle VC; the driving behavior information QJ includes the first driving behavior information QJ1 indicating the driving behavior while the driver DV is driving the vehicle VC and the second driving behavior information QJ2 indicating the driving behavior in the second period before the driver DV starts driving the vehicle VC; the danger level evaluation unit 114 evaluates the driving danger level DR based on the first biological information BJ1, the second biological information BJ2, the first driving behavior information QJ1, and the second driving behavior information QJ2; and the area setting unit 115 sets the danger area DA in the map information in accordance with the driving danger level DR.

Therefore, it is possible to appropriately evaluate the driving danger level DR, since the driving danger level DR is evaluated based on the first biological information BJ1, the second biological information BJ2, the first driving behavior information QJ1, and the second driving behavior information QJ2. It is also possible to appropriately set the danger area DA, since the danger area DA is set in the map information in accordance with the driving danger level DR.

In the smartphone 1, the driving history storage unit 120 is provided that stores the travel history HD of the vehicle VC of the driver DV, and when the travel history HD indicates that the danger area DA has been traveled in the past and appropriate driving behavior has been demonstrated in the danger area DA, the notification unit 117 does not output the first notification NT1 relating to the danger area DA.

Therefore, it is possible to suppress outputting unnecessary notification, since the first notification NT1 relating to the danger area DA is not outputted when the danger area DA has been traveled in the past and appropriate driving behavior has been demonstrated in the danger area DA.

In the smartphone 1, the notification unit 117 displays the danger area DA on the map in the form that corresponds to the danger degree of the danger area DA.

Thus, the driver DV can visually confirm the danger degree of the danger area DA, since the danger area DA is displayed on the map in the form that corresponds to the danger degree of the danger area DA. Therefore, it is possible to improve user-friendliness for the driver DV.

In the smartphone 1, the health state evaluation unit 112 is provided that evaluates the health state of the driver DV based on the first biological information BJ1 and the second biological information BJ2, the health state evaluation unit 112 evaluates whether the health state of the driver DV is an appropriate state based on each of a plurality of ability categories constituting the driving ability AB, and the notification unit 117 outputs, as the first notification NT1, the advice information AD relating to an ability category evaluated to be the lowest by the health state evaluation unit 112.

Therefore, it is possible to appropriately output the advice information AD, since the advice information AD relating to the ability category evaluated to be the lowest is output.

In the smartphone 1, when the travel position PS approaches the danger area DA, the notification unit 117 displays the warning information relating to the danger area DA.

Thus, the driver DV can visually confirm the warning information relating to the danger area DA, since the warning information relating to the danger area DA is displayed when the travel position PS approaches the danger area DA. Therefore, it is possible to improve user-friendliness for the driver DV.

The driving assistance method according to the present embodiment includes: the biological information acquisition step of acquiring the biological information BJ about the driver DV; the driving behavior acquisition step of acquiring the driving behavior information QJ indicating the driving behavior of the driver DV; the danger level evaluation step of evaluating the driving danger level DR indicating the danger degree while the driver DV is driving the vehicle VC, based on the biological information BJ and the driving behavior information QJ; the area setting step of setting the danger area DA in the map information MP stored in advance, when the driving danger level DR is equal to or higher than the predetermined value; the position acquisition step of acquiring the travel position PS of the vehicle VC on the map, based on the position information of the vehicle VC; and the notification step of outputting, when the travel position PS approaches the danger area DA, the first notification NT1 indicating that the travel position PS has approached the danger area DA.

The driving assistance method according to the present embodiment has a configuration similar to that of the smartphone 1 according to the present embodiment, and thus exhibits operations and effects similar to those of the smartphone 1 according to the present embodiment.

The first control program PG1 according to the present embodiment causes the first processor 11A included in the driving assistance apparatus to function as: the biological information acquisition unit 111 that acquires the biological information BJ about the driver DV; the driving behavior acquisition unit 113 that acquires the driving behavior information QJ indicating the driving behavior of the driver DV; the danger level evaluation unit 114 that evaluates the driving danger level DR indicating the danger degree while the driver DV is driving the vehicle VC, based on the biological information BJ and the driving behavior information QJ; the map information storage unit 110 that stores the map information MP; the area setting unit 115 that sets the danger area DA in the map information MP when the driving danger level DR is equal to or higher than the predetermined value; the position acquisition unit 116 that acquires the travel position PS of the vehicle VC on the map, based on the position information of the vehicle VC; and the notification unit 117 that outputs, when the travel position PS approaches the danger area DA, the first notification NT1 indicating that the travel position PS has approached the danger area DA.

The first control program PG1 according to the present embodiment has a configuration similar to that of the smartphone 1 according to the present embodiment, and thus exhibits operations and effects similar to those of the smartphone 1 according to the present embodiment.

The driving assistance system according to the present embodiment includes: the smartwatch 2 that detects the biological information BJ about the driver DV; the in-vehicle apparatus 3 that detects the driving behavior of the driver DV; and the smartphone 1 that communicates with the smartwatch 2 and the in-vehicle apparatus 3, in which the smartphone 1 includes: the biological information acquisition unit 111 that acquires the biological information BJ about the driver DV; the driving behavior acquisition unit 113 that acquires the driving behavior information QJ indicating the driving behavior of the driver DV; the danger level evaluation unit 114 that evaluates the driving danger level DR indicating the danger degree while the driver DV is driving the vehicle VC, based on the biological information BJ and the driving behavior information QJ; the map information storage unit 110 that stores the map information MP; the area setting unit 115 that sets the danger area DA in the map information MP when the driving danger level DR is equal to or higher than the predetermined value; the position acquisition unit 116 that acquires the travel position PS of the vehicle VC on the map, based on the position information of the vehicle VC; and the notification unit 117 that outputs, when the travel position PS approaches the danger area DA, the first notification NT1 indicating that the travel position PS has approached the danger area DA.

The driving assistance system 100 according to the present embodiment has a configuration similar to that of the smartphone 1 according to the present embodiment, and thus exhibits operations and effects similar to those of the smartphone 1 according to the present embodiment.

### 9. Other Embodiments

The embodiment described above is a preferred embodiment of the present invention. However, the present invention is not limited to the embodiment described above, and can be implemented with a variety of modifications within the scope or the spirit of the present invention.

In the present embodiment, a case is described in which the “driving assistance apparatus” is implemented as the smartphone 1, but the embodiment is not limited thereto. The “driving assistance apparatus” may, for example, be implemented as a personal computer, a tablet apparatus, a server apparatus, or the like. At least part of the “driving assistance apparatus” may be implemented as the ECU 31 of the in-vehicle apparatus 3.

In the present embodiment, a case is described in which the smartwatch 2 generates the biological information about the driver DV, but the embodiment is not limited thereto. The “device” that generates the biological information about the driver DV may be worn by the driver DV and may generate the biological information BJ.

For example, the “device” that generates the biological information about the driver DV may be worn on an arm, the head, a leg, or the like of the driver DV. For example, the “device” may be smart innerwear attached to innerwear worn by the driver DV. For example, the “device” may be a sensing device attached to the skin, inside the body, or the like of the driver DV.

In the present embodiment, a case is described in which the notification unit 117 outputs speech indicating the first notification NT1 from the speaker 16, but the embodiment is not limited thereto. The notification unit 117 may output the speech indicating the first notification NT1 from at least one of the left speaker 333a or the right speaker 333b of the vehicle VC.

In the present embodiment, a case is described in which the notification unit 117 displays an image indicating the first notification NT1 on the first touch panel 14, but the embodiment is not limited thereto. The notification unit 117 may display the image indicating the first notification NT1 on a fourth touch panel 44 included in the in-vehicle apparatus 3.

For example, each of the configurations of the smartwatch 2 shown in FIG. 2, the in-vehicle apparatus 3 shown in FIG. 3, and the smartphone 1 shown in FIG. 5 indicates a functional configuration, and the specific embodiment thereof is not particularly limited. In other words, hardware individually corresponding to the respective functional units does not necessarily need to be implemented, and it is possible to adopt a configuration in which one processor executes a program to realize functions of a plurality of functional units. Part of functions realized by software in the above embodiments may be realized by hardware or part of functions realized by hardware may be realized by software.

The units of processing of the flowchart shown in FIG. 8 are divided in accordance with the main processing content in order to facilitate understanding of the processing of the smartphone 1, but the present invention is not limited by the division method, naming, or the like of the units of processing shown in the flowchart of FIG. 8. The processing of the smartphone 1 can be further divided into more units of processing in accordance with the content of the processing, or one unit of processing can be further divided to include more processing. The processing order of the above flowchart is also not limited to the illustrated example.

### 10. Configurations Supported by the Above Embodiments

The above embodiments are specific examples of the following configurations.

### Configuration 1

A driving assistance apparatus includes: a biological information acquisition unit configured to acquire biological information about a user; a driving behavior acquisition unit configured to acquire driving behavior information indicating driving behavior of the user; a danger level evaluation unit configured to evaluate a driving danger level indicating a danger degree while the user is driving a vehicle, based on the biological information and the driving behavior information; a map information storage unit configured to store map information; an area setting unit configured to set a danger area in the map information when the driving danger level is equal to or higher than a predetermined value; a position acquisition unit configured to acquire a travel position of the vehicle on a map, based on position information of the vehicle; and a notification unit configured to output, when the travel position approaches the danger area, a first notification indicating that the travel position has approached the danger area.

The driving assistance apparatus of Configuration 1 can appropriately set the danger area, since the danger area is set in the map information when the driving danger level is equal to or higher than the predetermined value. The driving assistance apparatus according to Configuration 1 can also appropriately output the first notification, since the first notification indicating that the travel position has approached the danger area is output when the travel position approaches the danger area.

### Configuration 2

The driving assistance apparatus according to Configuration 1 further includes a vehicle speed acquisition unit configured to acquire vehicle speed information indicating a travel speed of the vehicle, in which the notification unit outputs a second notification being a more urgent notification than the first notification, when the travel speed is higher than a reference speed of a road on which the vehicle is traveling.

The driving assistance apparatus of Configuration 2 can appropriately output the second notification, since the second notification being a more urgent notification than the first notification is output when the travel speed is higher than the reference speed of the road on which the vehicle is traveling.

### Configuration 3

The driving assistance apparatus according to Configuration 1 or 2, in which the biological information includes first biological information being biological information about the user who is driving the vehicle and second biological information being biological information about the user who is in a first period before starting driving the vehicle, the driving behavior information includes first driving behavior information indicating driving behavior of the user who is driving the vehicle and second driving behavior information indicating driving behavior of the user who is in a second period before starting driving the vehicle, the danger level evaluation unit evaluates the driving danger level based on the first biological information, the second biological information, the first driving behavior information, and the second driving behavior information, and the area setting unit sets a danger area in the map information in accordance with the driving danger level.

The driving assistance apparatus of Configuration 3 can appropriately evaluate the driving danger level, since the driving danger level is evaluated based on the first biological information, the second biological information, the first driving behavior information, and the second driving behavior information. The driving assistance apparatus of Configuration 3 can also appropriately set the danger area, since the danger area is set in the map information in accordance with the driving danger level.

### Configuration 4

The driving assistance apparatus according to any one of Configurations 1 to 3 further includes a travel history storage unit configured to store a travel history of the vehicle of the user, in which when the travel history indicates that the danger area has been traveled in the past and appropriate driving behavior has been demonstrated in the danger area, the notification unit does not output the first notification relating to the danger area.

The driving assistance apparatus of Configuration 4 can suppress outputting unnecessary notification, since the first notification relating to the danger area is not outputted when the danger area has been traveled in the past and appropriate driving behavior has been demonstrated in the danger area.

### Configuration 5

The driving assistance apparatus according to any one of Configurations 1 to 4, in which the notification unit displays the danger area on the map in a form that corresponds to a danger level of the danger area.

In the driving assistance apparatus of Configuration 5, the driver can visually confirm the danger level of the danger area, since the danger area is displayed on the map in the form that corresponds to the danger level of the danger area. Therefore, it is possible to improve user-friendliness for the driver.

### Configuration 6

The driving assistance apparatus according to Configuration 3 further includes a health state evaluation unit configured to evaluate a health state of the user based on the first biological information and the second biological information, in which the health state evaluation unit evaluates whether the health state of the user is an appropriate state, based on each of a plurality of ability categories constituting a driving ability, and the notification unit outputs, as the first notification, advice information relating to an ability category evaluated to be the lowest by the health state evaluation unit.

The driving assistance apparatus of Configuration 6 can appropriately output the advice information, since the advice information relating to the ability category evaluated to be the lowest is output.

### Configuration 7

The driving assistance apparatus according to any one of Configurations 1 to 6, in which the notification unit displays, when the travel position approaches the danger area, warning information relating to the danger area.

In the driving assistance apparatus of Configuration 7, the driver can visually confirm the warning information relating to the danger area, since the warning information relating to the danger area is displayed when the travel position approaches the danger area. Therefore, it is possible to improve user-friendliness for the driver.

### Configuration 8

A driving assistance method includes: a biological information acquisition step of acquiring biological information about a user; a driving behavior acquisition step of acquiring driving behavior information indicating driving behavior of the user; a danger level evaluation step of evaluating a driving danger level indicating a danger degree while the user is driving a vehicle, based on the biological information and the driving behavior information; an area setting step of setting a danger area in map information stored in advance, when the driving danger level is equal to or higher than a predetermined value; a position acquisition step of acquiring a travel position of the vehicle on a map, based on position information of the vehicle; and a notification step of outputting, when the travel position approaches the danger area, a first notification indicating that the travel position has approached the danger area.

The driving assistance method of Configuration 8 has a configuration similar to that of the driving assistance apparatus of Configuration 1, and thus exhibits operations and effects similar to those of the driving assistance apparatus of Configuration 1.

### Configuration 9

A non-transitory computer readable storage medium storing a program causes a processor included in a driving assistance apparatus to function as: a biological information acquisition unit configured to acquire biological information about a user; a driving behavior acquisition unit configured to acquire driving behavior information indicating driving behavior of the user; a danger level evaluation unit configured to evaluate a driving danger level indicating a danger degree while the user is driving a vehicle, based on the biological information and the driving behavior information; a map information storage unit configured to store map information; an area setting unit configured to set a danger area in the map information when the driving danger level is equal to or higher than a predetermined value; a position acquisition unit configured to acquire a travel position of the vehicle on a map, based on position information of the vehicle; and a notification unit configured to output, when the travel position approaches the danger area, a first notification indicating that the travel position has approached the danger area.

The storage medium of Configuration 9 has a configuration similar to that of the driving assistance apparatus of Configuration 1, and thus exhibits operations and effects similar to those of the driving assistance apparatus of Configuration 1.

### Configuration 10

A driving assistance system includes: a biological detection apparatus configured to detect biological information about a user; a driving detection apparatus configured to detect driving behavior of the user; and a driving assistance apparatus configured to communicate with the biological detection apparatus and the driving detection apparatus, in which the driving assistance apparatus includes: a biological information acquisition unit configured to acquire the biological information about the user from the biological detection apparatus; a driving behavior acquisition unit configured to acquire driving behavior information indicating the driving behavior of the user from the driving detection apparatus; a danger level evaluation unit configured to evaluate a driving danger level indicating a danger degree while the user is driving a vehicle, based on the biological information and the driving behavior information; a map information storage unit configured to store map information; an area setting unit configured to set a danger area in the map information when the driving danger level is equal to or higher than a predetermined value; a position acquisition unit configured to acquire a travel position of the vehicle on a map, based on position information of the vehicle; and a notification unit configured to output, when the travel position approaches the danger area, a first notification indicating that the travel position has approached the danger area.

The driving assistance system of Configuration 10 has a configuration similar to that of the driving assistance apparatus of Configuration 1, and thus exhibits operations and effects similar to those of the driving assistance apparatus of Configuration 1.

### REFERENCE SIGNS LIST

100 driving assistance system
1 smartphone (driving assistance apparatus)
11 first control unit
11A first processor
11B first memory
110 map information storage unit
111 biological information acquisition unit
112 health state evaluation unit
113 driving behavior acquisition unit
114 danger level evaluation unit
115 area setting unit
116 position acquisition unit
117 notification unit
118 vehicle speed acquisition unit
120 driving history storage unit
16 speaker
17 microphone
14 first touch panel
2 smartwatch (biological detection apparatus)
25 biological information generation unit
3 in-vehicle apparatus (driving detection apparatus)
31 ECU
35 driving information generation unit
AB driving ability
AB1 planning ability
AB2 perception ability
AB3 attention ability
AB4 judgment ability
AB5 operation ability
AD advice information
BJ biological information
BJ1 first biological information
BJ11 first sleep information
BJ12 first exercise information
BJ13 first stress information
BJ2 second biological information
BJ21 second sleep information
BJ22 second exercise information
BJ23 second stress information
DR driving danger level
DR1 first driving danger level
DR2 second driving danger level
DV driver (user)
HD travel history
MP map information
NT1 first notification
NT2 second notification
PG1 first control program (driving assistance program)
PS travel position
QJ driving information
QJ1 first driving information
QJ2 second driving information
RS health rating
RS1 planning ability rating
RS2 perception ability rating
RS3 attention ability rating
RS4 judgment ability rating
RS5 operation ability rating
VA reference speed
VC vehicle
VS travel speed

1. A driving assistance apparatus, comprising:
a biological information acquisition unit configured to acquire biological information about a user;
a driving behavior acquisition unit configured to acquire driving behavior information indicating driving behavior of the user;
a danger level evaluation unit configured to evaluate a driving danger level indicating a danger degree while the user is driving a vehicle, based on the biological information and the driving behavior information;
a map information storage unit configured to store map information;
an area setting unit configured to set a danger area in the map information when the driving danger level is equal to or higher than a predetermined value;
a position acquisition unit configured to acquire a travel position of the vehicle on a map, based on position information of the vehicle; and
a notification unit configured to output, when the travel position approaches the danger area, a first notification indicating that the travel position has approached the danger area.

2. The driving assistance apparatus according to claim 1, further comprising a vehicle speed acquisition unit configured to acquire vehicle speed information indicating a travel speed of the vehicle, wherein
the notification unit outputs a second notification being a more urgent notification than the first notification, when the travel speed is higher than a reference speed of a road on which the vehicle is traveling.

3. The driving assistance apparatus according to claim 1, wherein
the biological information includes first biological information being biological information about the user who is driving the vehicle and second biological information being biological information about the user who is in a first period before starting driving the vehicle,
the driving behavior information includes first driving behavior information indicating driving behavior of the user who is driving the vehicle and second driving behavior information indicating driving behavior of the user who is in a second period before starting driving the vehicle,
the danger level evaluation unit evaluates the driving danger level based on the first biological information, the second biological information, the first driving behavior information, and the second driving behavior information, and
the area setting unit sets a danger area in the map information in accordance with the driving danger level.

4. The driving assistance apparatus according to claim 1, further comprising a travel history storage unit configured to store a travel history of the vehicle of the user, wherein
when the travel history indicates that the danger area has been traveled in the past and appropriate driving behavior has been demonstrated in the danger area, the notification unit does not output the first notification relating to the danger area.

5. The driving assistance apparatus according to claim 1, wherein the notification unit displays the danger area on the map in a form that corresponds to a danger level of the danger area.

6. The driving assistance apparatus according to claim 3, comprising a health state evaluation unit configured to evaluate a health state of the user based on the first biological information and the second biological information, wherein
the health state evaluation unit evaluates whether the health state of the user is an appropriate state, based on each of a plurality of ability categories constituting a driving ability, and
the notification unit outputs, as the first notification, advice information relating to an ability category evaluated to be the lowest by the health state evaluation unit.

7. The driving assistance apparatus according to claim 1, wherein the notification unit displays, when the travel position approaches the danger area, warning information relating to the danger area.

8. A driving assistance method, comprising:
a biological information acquisition step of acquiring biological information about a user;
a driving behavior acquisition step of acquiring driving behavior information indicating driving behavior of the user;
a danger level evaluation step of evaluating a driving danger level indicating a danger degree while the user is driving a vehicle, based on the biological information and the driving behavior information;
an area setting step of setting a danger area in map information stored in advance, when the driving danger level is equal to or higher than a predetermined value;
a position acquisition step of acquiring a travel position of the vehicle on a map, based on position information of the vehicle; and
a notification step of outputting, when the travel position approaches the danger area, a first notification indicating that the travel position has approached the danger area.

9. A non-transitory computer readable storage medium storing a driving assistance program for causing a processor included in a driving assistance apparatus to function as:
a biological information acquisition unit configured to acquire biological information about a user;
a driving behavior acquisition unit configured to acquire driving behavior information indicating driving behavior of the user;
a danger level evaluation unit configured to evaluate a driving danger level indicating a danger degree while the user is driving a vehicle, based on the biological information and the driving behavior information;
a map information storage unit configured to store map information;
an area setting unit configured to set a danger area in the map information when the driving danger level is equal to or higher than a predetermined value;
a position acquisition unit configured to acquire a travel position of the vehicle on a map, based on position information of the vehicle; and
a notification unit configured to output, when the travel position approaches the danger area, a first notification indicating that the travel position has approached the danger area.

10. A driving assistance system, comprising:
a biological detection apparatus configured to detect biological information about a user;
a driving detection apparatus configured to detect driving behavior of the user; and
a driving assistance apparatus configured to communicate with the biological detection apparatus and the driving detection apparatus, wherein
the driving assistance apparatus includes:
a biological information acquisition unit configured to acquire the biological information about the user from the biological detection apparatus;
a driving behavior acquisition unit configured to acquire driving behavior information indicating the driving behavior of the user from the driving detection apparatus;
a danger level evaluation unit configured to evaluate a driving danger level indicating a danger degree while the user is driving a vehicle, based on the biological information and the driving behavior information;
a map information storage unit configured to store map information;
an area setting unit configured to set a danger area in the map information when the driving danger level is equal to or higher than a predetermined value;
a position acquisition unit configured to acquire a travel position of the vehicle on a map, based on position information of the vehicle; and
a notification unit configured to output, when the travel position approaches the danger area, a first notification indicating that the travel position has approached the danger area.
